(19) United States
(12) Patent Application Publication
(10) Pub. No.: US 2025/0248413 A1
(43) Pub. Date: Aug. 7, 2025

(54) MONITORING SYSTEM OF HEAT PUMP TYPE LOW-TEMPERATURE CIRCULATING GRAIN DRYER BASED ON INTERNET OF THINGS

(71) Applicant: NANJING AGRICULTURAL UNIVERSITY, Nanjing City (CN)

(72) Inventors: Kunjie CHEN, Nanjing (CN); Jichao HUANG, Nanjing (CN); Zixuan CHEN, Nanjing (CN); Yi ZUO, Nanjing (CN)

(21) Appl. No.: 18/674,827

(22) Filed: May 25, 2024

(30) Foreign Application Priority Data
Feb. 1, 2024 (CN) 202410143226.0

(51) Int. Cl. A23B 9/08; F26B 21/04; F26B 21/08; F26B 21/10

(52) U.S. Cl. CPC A23B 9/08; F26B 21/04; F26B 21/086; F26B 21/10; F26B 2200/06

(57) ABSTRACT

The application discloses a monitoring system of a heat pump type low-temperature circulating grain dryer based on the Internet of Things, which includes a data acquisition module, a data transmission module, a safety control module and a remote monitoring module; where the data acquisition module is used for obtaining data of a low-temperature circulating dryer and a multistage air source heat pump; the safety control module is used for automatically controlling a motor according to the data; the data transmission module is used for transmitting data to the cloud server; the remote monitoring module is used for graphically displaying the data, performing an alarming and sending control commands on the low-temperature circulating dryer and the multistage air source heat pump; and the data acquisition module, the safety control module and the data transmission module are wirelessly connected with the remote monitoring module.

### CROSS-REFERENCE TO RELATED APPLICATIONS

This application claims priority to Chinese Patent Application No. 202410143226.0, filed on Feb. 1, 2024, the contents of which are hereby incorporated by reference.

### TECHNICAL FIELD

The application belongs to the technical field of monitoring systems of grain dryers, and in particular to a monitoring system of a heat pump type low-temperature circulating grain dryer based on the Internet of Things.

### BACKGROUND

With the implementation of environmental protection and dual carbon policies, equipment with heat source such as heat pump, infrared, geothermal energy and microwave is more and more used in grain drying, among which the low-temperature circulating dryer with air source heat pump as heat source is widely popularized and applied. However, at present, the monitoring of the dryer itself lacks the control of the heat source equipment, and there is a gap regarding the remote monitoring of the heat pump type low-temperature circulating dryer.

### SUMMARY

In order to solve the above technical problems, the application provides a monitoring system of a heat pump type low-temperature circulating grain dryer based on the Internet of Things, aiming at realizing remote centralized control, saving manpower and reducing potential safety hazards of equipment.

In order to achieve the above objectives, the present application provides a monitoring system of a heat pump type low-temperature circulating grain dryer based on the Internet of Things, including:
a data acquisition module, a data transmission module, a safety control module and a remote monitoring module;
where the data acquisition module is used for obtaining data of a low-temperature circulating dryer and a multistage air source heat pump;
the safety control module is used for automatically controlling a motor according to the data;
the data transmission module is used for transmitting the data to the remote monitoring module;
the remote monitoring module is used for graphically displaying the data, performing an alarming and sending control commands on the low-temperature circulating dryer and the multistage air source heat pump; and
the data acquisition module, the safety control module and the data transmission module are wirelessly connected with the remote monitoring module.

Optionally, the data acquisition module includes a data acquisition unit of the low-temperature circulating dryer and a data acquisition unit of a multistage air source heat pump;
where the data acquisition unit of the low-temperature circulating dryer is used for monitoring a moisture value, a drying temperature, hot wind temperature and humidity data and wind speed data of grains in the low-temperature circulating dryer; and
the data acquisition unit of the multistage air source heat pump is used for monitoring data of a compressor and an evaporator of the multistage air source heat pump and environmental data.

Optionally, the data acquisition unit of the low-temperature circulating dryer includes a temperature sensor, a wind speed sensor, a temperature and humidity sensor and a moisture meter;
where the temperature sensor is used for collecting drying temperature data in the low-temperature circulating dryer;
the wind speed sensor is used for collecting the wind speed data of hot wind;
the temperature and humidity sensor is used for collecting the hot wind temperature and humidity data in a hot wind pipeline; and
the moisture meter is used for detecting moisture in the low-temperature circulating dryer and obtaining the moisture value of the grains in a drying bin.

Optionally, obtaining the moisture value of the grains in the drying bin includes:
S1, sending a code of dry variety to the moisture meter by a controller, and receiving the code and returning a receiving success signal by the moisture meter, and transmitting a polling signal to S2, and if a dry variety signal fails to be received, repeating the S1;
S2, sending a moisture setting command to the moisture meter by the controller, receiving the moisture setting command and returning the receiving success signal by the moisture meter, and transmitting the polling signal to S3, and if the moisture setting command fails to be received, repeating the S2;
S3, sending a moisture detecting command to the moisture meter by the controller, and receiving the moisture detecting command and returning a receiving success signal by the moisture meter, and transmitting the polling signal to S4, and if the moisture detecting command fails to be received, repeating the S3;
S4, detecting the moisture value of the grains by the moisture meter, and judging whether a moisture detecting is completed according to a running state; if the moisture detecting is completed, returning the data to the controller, the controller judges whether the moisture value of the grains is valid; if the moisture value of the grains is invalid, the system fails; if the moisture value of the grains is valid, obtaining the moisture value of the grains.

Optionally, the data acquisition unit of the multistage air source heat pump includes a temperature sensor group and a pressure sensor;
where the temperature sensor group is used for obtaining outlet exhaust temperature data of the compressor, pipeline fin temperature data of the evaporator and ambient wind temperature data of an air inlet pipe intersection; and
the pressure sensor is used for obtaining pressure data of the compressor of the multistage air source heat pump.

Optionally, the safety control module includes a thermal protection unit, a wind volume protection unit and a compressor high and low pressure protection unit;
where the thermal protection unit is used for judging whether equipment is faulty according to a signal of a thermal protection relay, stopping the equipment if the equipment is faulty and sending fault information to the remote monitor module;
the wind volume protection unit is used for obtaining a wind volume value according to the wind speed data, sending insufficient wind volume information to the remote monitoring module according to the wind volume value, stopping the equipment and sending the fault information to the remote monitoring module; and
the compressor high and low pressure protection unit is used for turning off or turning on the compressor according to the pressure data of the compressor, and sending the fault information to the remote monitoring module when turn-off times of the compressor exceed a threshold.

Optionally, the data transmission module includes a 4G communication unit, a signal base station, a network cloud and a local server; the 4G communication unit, the signal base station, the network cloud and the local server are wirelessly connected.

Optionally, the remote monitoring module includes a touch screen and a mobile terminal;
where the touch screen is used for monitoring and controlling the low-temperature circulating dryer and the multistage air source heat pump; and
the mobile terminal is used for obtaining the data through the network cloud and performing a remote monitoring.

The technical effect of the present application are as follows: in the application, a user may realize real-time monitoring of the parameters of the multistage air source heat pump and the parameters of the low-temperature circulating dryer through the webpage terminal and the mobile phone APP terminal; the communication success rate of the system is higher than 98%, and the average absolute errors of real-time detection of ambient temperature, hot wind temperature, hot wind humidity and hot wind speed are 0.3° C., 0.26° C., 1.4% RH and 0.47 m/s respectively, which may meet the requirements of grain drying monitoring. The system may be used for actual production display, and has stable system operation, high degree of automation and comprehensive safety protection function, and may realize remote centralized control, save manpower, reduce potential safety hazards of equipment, and has good practicability and operability.

### BRIEF DESCRIPTION OF THE DRAWINGS

The accompanying drawings, which constitute a part of this application, are used to provide a further understanding of this application. The illustrative embodiments of this application and their descriptions are used to explain this application, and do not constitute an improper limitation of this application. In the attached drawings:

FIG. 1 is a schematic diagram of a monitoring system of a heat pump type low-temperature circulating grain dryer based on the Internet of Things according to an embodiment of the present application.

FIG. 2A is a schematic diagram of ambient temperature parameter detection error in an embodiment of the present application.

FIG. 2B is a schematic diagram of hot wind temperature parameter detection error in an embodiment of the present application.

FIG. 2C is a schematic diagram of hot wind humidity parameter detection error in an embodiment of the present application.

FIG. 2D is a schematic diagram of hot wind speed parameter detection error in an embodiment of the present application.

FIG. 3 is a schematic diagram of the steps for obtaining the moisture value of the grains in the drying bin.

### DETAILED DESCRIPTION OF THE EMBODIMENTS

It should be noted that the embodiments in this application and the features in the embodiments may be combined with each other without conflict. The present application will be described in detail with reference to the attached drawings and embodiments.

It should be noted that the steps shown in the flowchart of the accompanying drawings may be executed in a computer system such as a set of computer-executable instructions, and although the logical order is shown in the flowchart, in some cases, the steps shown or described may be executed in a different order from here.

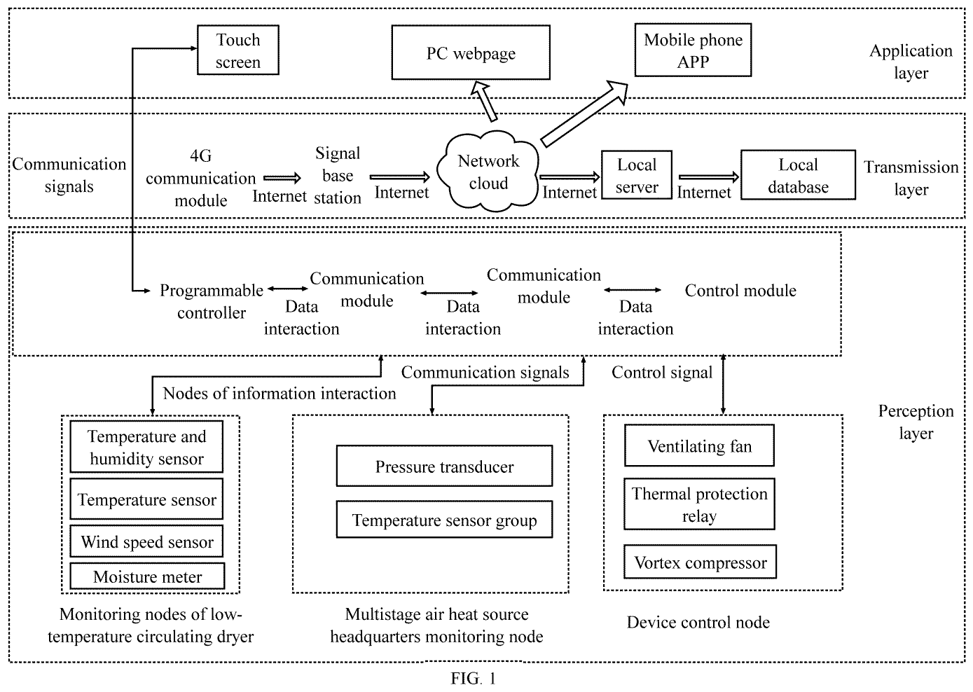

As shown in FIG. 1, a monitoring system of a heat pump type low-temperature circulating grain dryer based on the Internet of Things provided in this embodiment includes a data acquisition module, a data transmission module, a safety control module and a remote monitoring module;
where the data acquisition module is used for obtaining data of a low-temperature circulating dryer and a multistage air source heat pump;
the safety control module is used for automatically controlling a motor according to the data;
the data transmission module is used for transmitting the data to the remote monitoring module;
the remote monitoring module is used for graphically displaying the data, performing an alarming and sending control commands on the low-temperature circulating dryer and the multistage air source heat pump; and
the data acquisition module, the safety control module and the data transmission module are wirelessly connected with the remote monitoring module.
1) Perception layer (data acquisition module and safety control module): mainly including monitoring nodes of low-temperature circulating dryer, monitoring nodes of multistage air source heat pump, nodes of equipment control and nodes of information interaction. The sensor data of the nodes is transmitted to the communication module, the motor control signal is transmitted to the control module, the programmable controller integrates and packages the information and sends the information to the 4G communication module, and then the data is sent to the base station of the transmission layer through the network;
2) Transmission layer (data transmission module): mainly composed of 4G communication module, signal base station, network cloud and local server. The purpose is to transmit the data obtained by the perception layer to the cloud server through the network and download the data to the local server for storage.
3) Application layer (remote monitoring module): on-site touch screen may display the monitoring equipment (dryer and heat pump) graphically, give an alarming and issue control commands. Computer webpages and mobile phone APP obtain data through the interface of the cloud server, and provide remote monitoring services for subordinate users.

### Data Acquisition Module:

\textcircled{1} real-time monitoring of the low-temperature circulating dryer, monitoring the moisture value of grains in the drying bin, the temperature and wind speed of hot air, and the grain temperature in the drying bin. The temperature sensor is used for collecting drying temperature data in the low-temperature circulating dryer.

In this embodiment, the target drying temperature may be set, and the control of the heat pump may be adjusted and corrected according to the ambient temperature and the actual drying temperature.

The temperature and humidity sensor is used for collecting the temperature and humidity of the hot wind in the hot wind pipeline;
the moisture meter is used for detecting the moisture in the low-temperature circulating dryer and obtaining the moisture value of the grains in the drying bin.

The wind speed sensor is used for collecting the wind speed data of the hot air by collecting the wind speed in the hot wind pipeline.

\textcircled{2} Multistage air source heat pump real-time monitoring, monitoring the high and low pressure of compressor inlet and outlet pipelines and compressor outlet exhaust temperature of each heat pump system; evaporator pipeline fin temperature; ambient wind temperature at air inlet pipe intersection.

The pressure sensor is used to obtain the pressure data of the compressor of the multistage air source heat pump;
the temperature sensor group is used for collecting the inlet temperature of the multistage air source heat pump, and further collect the ambient temperature and the compressor outlet exhaust temperature and the evaporator pipeline fin temperature.

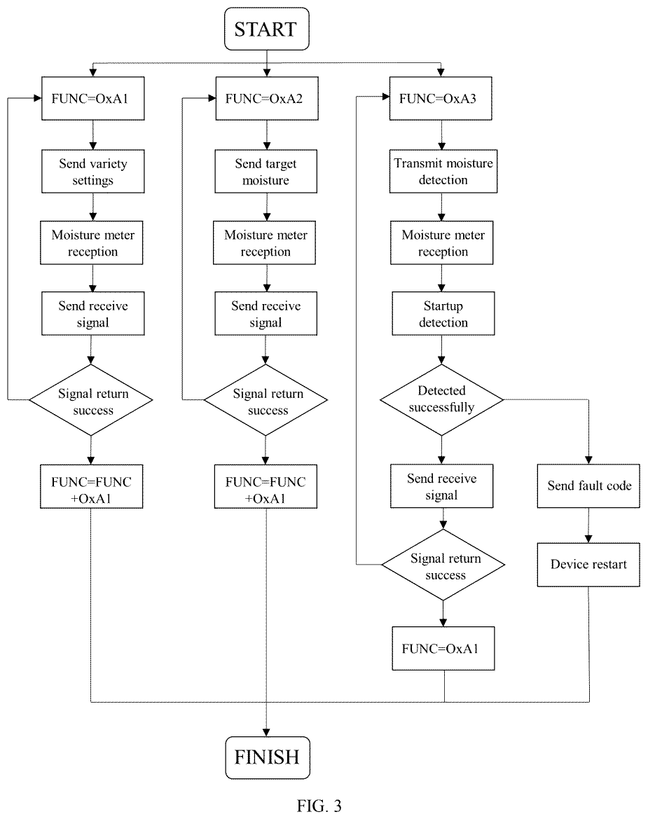

Obtaining the moisture value of the grains in the drying bin include the following steps (as shown in FIG. 3).
S1, sending a code of dry variety to the moisture meter by a controller, and receiving the code and returning a receiving success signal by the moisture meter, and transmitting a polling signal to S2, and if a dry variety signal fails to be received, repeating the S1;
S2, sending a moisture setting command to the moisture meter by the controller, receiving the moisture setting command and returning the receiving success signal by the moisture meter, and transmitting the polling signal to S3, and if the moisture setting command fails to be received, repeating the S2;
S3, sending a moisture detecting command to the moisture meter by the controller, and receiving the moisture detecting command and returning a receiving success signal by the moisture meter, and transmitting the polling signal to S4, and if the moisture detecting command fails to be received, repeating the S3;
S4, detecting the moisture value of the grains by the moisture meter, and judging whether a moisture detecting is completed according to a running state; if the moisture detecting is completed, returning the data to the controller, the controller judges whether the moisture value of the grains is valid; if the moisture value of the grains is invalid, the system fails; if the moisture value of the grains is valid, obtaining the moisture value of the grains. The end of moisture detecting is marked by the end signal automatically fed back by the moisture meter, and the data string fed back by the moisture meter contains “valid bit” data information, so it is judged that the moisture value of the grains is valid.

The polling mode avoids channel congestion and step confusion, and may be executed from any step down according to the requirements.

Safety control module: the heat pump type low-temperature circulating dryer is controlled automatically, the monitoring information is summarized and analyzed to realize automatic control and safety protection of each motor during grain drying. The operation state of the heat pump type low-temperature circulating dryer is monitored by protection units at different levels, which replaces the traditional manual monitoring of the drying process. System protection unit: \textcircled{1} thermal protection unit: the system immediately stops all equipment operation and sends fault information after receiving the signal of the thermal protection relay; (2) wind volume protection unit: the system calculates the wind volume Q per unit time according to the wind speed; if 7,200 m³/h<Q<12,000 m³/h, the insufficient wind volume information is sent; if Q<7,200 m³/h, the system immediately stops the operation of all equipment and sends the fault information; \textcircled{3} Compressor high and low pressure protection unit: when high pressure or low pressure is triggered, the corresponding compressor is turned off, and the compressor is restarted after the pressure returns to normal for 3 minutes. If the compressor stops for many times, the operation of the compressor is stopped and compressor fault information is sent.

Remote monitoring module: with multi-device operation function, and the staff may monitor and control the devices through the on-site touch screen. It is also possible to realize unmanned grain drying through remote monitoring on the equipment by the computer and mobile phone APP.

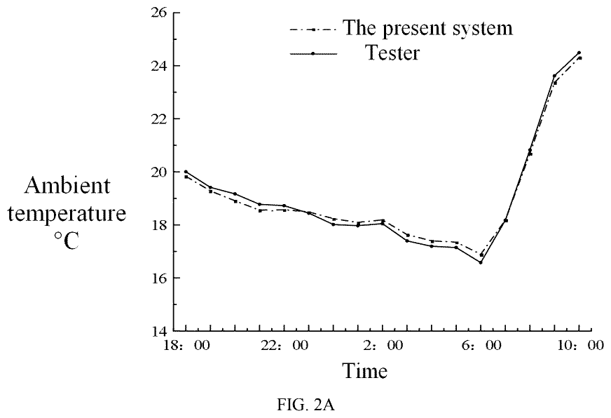

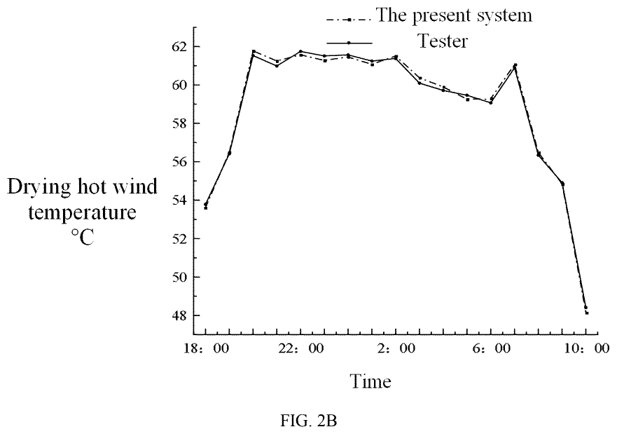

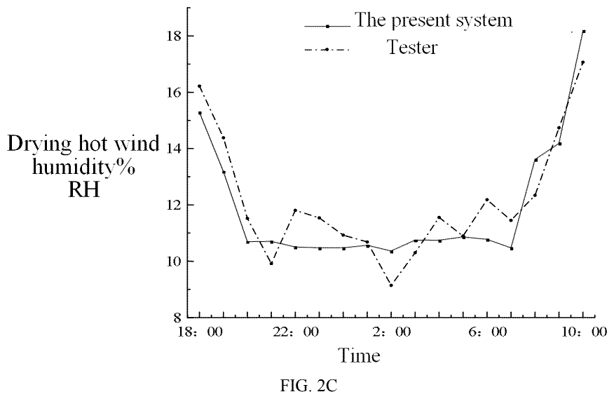

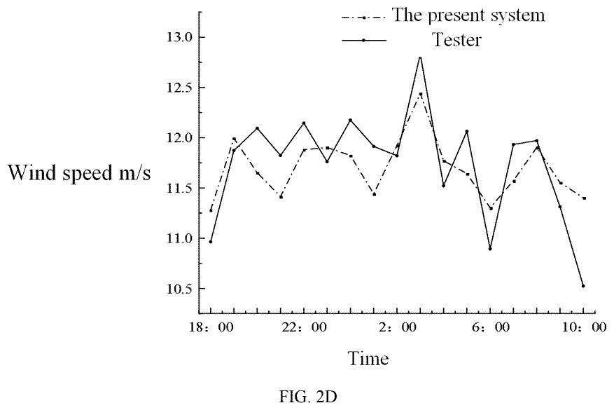

Taking the data measured by hand-held temperature and humidity detector and tsi9535-A hot-wire anemometer every 30 min as reference, the error test of four parameters, including ambient temperature, hot wind temperature, hot wind humidity and hot wind speed, is carried out, and the results are shown in FIG. 2A-FIG. 2D. FIG. 2A is the schematic diagram of ambient temperature parameter detection error, FIG. 2B is the schematic diagram of hot wind temperature parameter detection error, FIG. 2C is the schematic diagram of hot wind humidity parameter detection error and FIG. 2D is the schematic diagram of hot wind speed parameter detection error. The average absolute error of the ambient temperature detected by the system is 0.3° C., the error of the hot wind temperature is 0.26° C., the error of the hot wind humidity is 1.2% RH, and the error of the hot wind speed is 0.47 m/s. All the errors are very small, which may fully satisfy the use of grain drying and monitoring.

The 4G communication module is set to report data to the server once every 1 min, and the data of five rice drying experiments from October 15th to Nov. 15, 2022 are collected. During the total drying time of 80 h, the multistage air source heat pump monitoring point should report 115,200 pieces of data to the server, the low-temperature circulating dryer should report 72,000 pieces of data, and the equipment control node should report 52,800 pieces of data. The log of MYSQL database is read and analyzed, and the results are shown in Table 1. The results show that the average failure rate of system communication is about 1.76%, and the success rate is over 98%, which indicates that the system is stable.

TABLE 1

| | | Loss |
|---|---|---|
| Data source | Number | rate/% |
| Heat pump monitoring | 112 965 | 1.94 |
| point | | |
| Dryer monitoring point | 70 502 | 2.08 |
| Device control node | 52 140 | 1.25 |
| Average | — | 1.76 |

The above is only the preferred embodiment of this application, but the protection scope of this application is not limited to this. Any change or replacement that may be easily thought of by a person familiar with this technical field within the technical scope disclosed in this application should be included in the protection scope of this application. Therefore, the protection scope of this application should be based on the protection scope of the claims.

What is claimed is:

1. A monitoring system of a heat pump type low-temperature circulating grain dryer based on Internet of Things, comprising a data acquisition module, a data transmission module, a safety control module and a remote monitoring module;
wherein the data acquisition module is used for obtaining data of a low-temperature circulating dryer and a multistage air source heat pump;
the safety control module is used for automatically controlling a motor according to the data;
the data transmission module is used for transmitting the data to the remote monitoring module;
the remote monitoring module is used for graphically displaying the data, performing an alarming and sending control commands on the low-temperature circulating dryer and the multistage air source heat pump; and
the data acquisition module, the safety control module and the data transmission module are wirelessly connected with the remote monitoring module.

2. The monitoring system of the heat pump type low-temperature circulating grain dryer based on the Internet of Things according to claim 1, wherein the data acquisition module comprises a data acquisition unit of the low-temperature circulating dryer and a data acquisition unit of a multistage air source heat pump;
wherein the data acquisition unit of the low-temperature circulating dryer is used for monitoring a moisture value, a drying temperature, hot wind temperature and humidity data and wind speed data of grains in the low-temperature circulating dryer; and
the data acquisition unit of the multistage air source heat pump is used for monitoring data of a compressor and an evaporator of the multistage air source heat pump and environmental data.

3. The monitoring system of the heat pump type low-temperature circulating grain dryer based on the Internet of Things according to claim 2, wherein the data acquisition unit of the low-temperature circulating dryer comprises a temperature sensor, a wind speed sensor, a temperature and humidity sensor and a moisture meter;
wherein the temperature sensor is used for collecting drying temperature data in the low-temperature circulating dryer;
the wind speed sensor is used for collecting the wind speed data of hot wind;
the temperature and humidity sensor is used for collecting the hot wind temperature and humidity data in a hot wind pipeline; and
the moisture meter is used for detecting moisture in the low-temperature circulating dryer and obtaining the moisture value of the grains in a drying bin.

4. The monitoring system of the heat pump type low-temperature circulating grain dryer based on the Internet of Things according to claim 3, wherein obtaining the moisture value of the grains in the drying bin comprises:
S1, sending a code of dry variety to the moisture meter by a controller, and receiving the code and returning a receiving success signal by the moisture meter, and transmitting a polling signal to S2, and if a dry variety signal fails to be received, repeating the S1;
S2, sending a moisture setting command to the moisture meter by the controller, receiving the moisture setting command and returning the receiving success signal by the moisture meter, and transmitting the polling signal to S3, and if the moisture setting command fails to be received, repeating the S2;
S3, sending a moisture detecting command to the moisture meter by the controller, and receiving the moisture detecting command and returning a receiving success signal by the moisture meter, and transmitting the polling signal to S4, and if the moisture detecting command fails to be received, repeating the S3;
S4, detecting the moisture value of the grains by the moisture meter, and judging whether a moisture detecting is completed according to a running state; if the moisture detecting is completed, returning the data to the controller, the controller judges whether the moisture value of the grains is valid; if the moisture value of the grains is invalid, the system fails; if the moisture value of the grains is valid, obtaining the moisture value of the grains.

5. The monitoring system of the heat pump type low-temperature circulating grain dryer based on the Internet of Things according to claim 2, wherein the data acquisition unit of the multistage air source heat pump comprises a temperature sensor group and a pressure sensor;
wherein the temperature sensor group is used for obtaining outlet exhaust temperature data of the compressor, pipeline fin temperature data of the evaporator and ambient wind temperature data of an air inlet pipe intersection; and
the pressure sensor is used for obtaining pressure data of the compressor of the multistage air source heat pump.

6. The monitoring system of the heat pump type low-temperature circulating grain dryer based on the Internet of Things according to claim 5, wherein the safety control module comprises a thermal protection unit, a wind volume protection unit and a compressor high and low pressure protection unit;
wherein the thermal protection unit is used for judging whether an equipment is faulty according to a signal of a thermal protection relay, stopping the equipment if the equipment is faulty and sending fault information to the remote monitor module;
the wind volume protection unit is used for obtaining a wind volume value according to the wind speed data, sending insufficient wind volume information to the remote monitoring module according to the wind volume value, stopping the equipment and sending the fault information to the remote monitoring module; and
the compressor high and low pressure protection unit is used for turning off or turning on the compressor according to the pressure data of the compressor, and sending the fault information to the remote monitoring module when turn-off times of the compressor exceed a threshold.

7. The monitoring system of the heat pump type low-temperature circulating grain dryer based on the Internet of Things according to claim 1, wherein the data transmission module comprises a 4G communication unit, a signal base station, a network cloud and a local server; the 4G communication unit, the signal base station, the network cloud and the local server are wirelessly connected.

8. The monitoring system of the heat pump type low-temperature circulating grain dryer based on the Internet of Things according to claim 7, wherein the remote monitoring module comprises a touch screen and a mobile terminal;
the touch screen is used for monitoring and controlling the low-temperature circulating dryer and the multistage air source heat pump; and
the mobile terminal is used for obtaining the data through the network cloud and performing a remote monitoring.
